(19) United States
(12) Patent Application Publication
(10) Pub. No.: US 2024/0324968 A1
(43) Pub. Date: Oct. 3, 2024

(54) WEARABLE INCONTINENCE PREDICTOR SYSTEMS AND DEVICES, AND METHODS FOR PREDICTING INCONTINENCE

(71) Applicant: Welch Allyn, Inc., Skaneateles Falls, NY (US)

(72) Inventors: Prathamesh Kharkar, Skaneateles Falls, NY (US); Christopher Nelson, Skaneateles Falls, NY (US)

(73) Assignee: Welch Allyn, Inc., Skaneateles Falls, NY (US)

(21) Appl. No.: 18/621,245

(22) Filed: Mar. 29, 2024

Related U.S. Application Data
(60) Provisional application No. 63/493,515, filed on Mar. 31, 2023.

(51) Int. Cl. A61B 5/00; A61B 5/0536; A61B 5/20

(52) U.S. Cl. CPC A61B 5/7275; A61B 5/0536; A61B 5/204; A61B 5/4561; A61B 5/6808; A61B 5/7282

(57) ABSTRACT

A system for incontinence prediction includes an electrical impedance tomography (EIT) device, a posture detector, and one or more processors communicatively coupled to the EIT device and the posture detector. The EIT device may include a plurality of electrodes configured to be worn at a lower abdomen of a user around the bladder. The one or more processors is configured to collect an EIT image generated by the EIT device, a posture generated by the posture detector, and a planning activity of the user. The one or more processors may further determine a bladder status based on the EIT image of the user, predict an incontinence risk based on the bladder status, the posture, and the planning activity of the user and determine a high-risk point indicating a high incontinence risk, and notify the user or a caregiver of the high incontinence risk at the high-risk point.

### CROSS-REFERENCE TO RELATED APPLICATION

The present application claims the priority benefit of U.S. Provisional Application Ser. No. 63/493,515, entitled “WEARABLE INCONTINENCE PREDICTOR SYSTEMS AND DEVICES, AND METHODS FOR PREDICTING INCONTINENCE” and filed Mar. 31, 2023, the entire contents of which are incorporated herein by reference.

### TECHNICAL FIELD

The present disclosure relates to subject monitoring systems, and more particularly, to incontinence prediction systems.

### BACKGROUND

Currently, an estimated 423 million people (20 years and older) worldwide experience some form of urinary incontinence with approximately 13 million Americans experiencing urinary incontinence. The prevalence is 50% or greater among residents of nursing facilities. Caregivers report that 53% of the homebound elderly are incontinent. People who have symptoms of unintentional loss of urine often feel embarrassed or ashamed about their conditions. Incontinence further causes problems, such as skin irritation and infection, sleep disruption, and increased risk of falls, which influence a person's quality of life. Accordingly, there is a need for wearable incontinence predictor device for predicting incontinence.

### SUMMARY

In a first aspect, a system for incontinence prediction includes an electrical impedance tomography (EIT) device that includes a plurality of electrodes configured to be worn at a lower abdomen of a user around the bladder. A first two or more electrodes are configured to apply alternating currents to a body of the user, and a second two or more electrodes record resulting potentials detected in the body of the user. The system further includes a posture detector and one or more processors communicatively coupled to the EIT device and the posture detector. The one or more processors are configured to collect an EIT image generated by the EIT device, a posture generated by the posture detector, and a planning activity of the user, determine a bladder status based on the EIT image of the user, predict an incontinence risk based on the bladder status, the posture, and the planning activity of the user and determine a high-risk point indicating a high incontinence risk, and notify the user or a caregiver of the high incontinence risk at the high-risk point.

In a second aspect, a garment for incontinence prediction includes a garment body formed of a flexible fabric. The garment body is configured to be worn on a lower torso of a user. The garment further includes an EIT device. The EIT device includes a plurality of electrodes arranged at discrete locations on the garment body. Each electrode is positioned on an inner surface of the garment body and is configured to contact at a lower abdomen of the user around the bladder. The garment also includes a posture detector arranged at an inner surface of the garment body, and one or more processors communicatively coupled to the EIT device and the posture detector. The one or more processors are configured to collect an EIT image generated by the EIT device, a posture generated by the posture detector, and a planning activity of the user, determine a bladder status based on the EIT image of the user, predict an incontinence risk based on the bladder status, the posture, and the planning activity of the user, and determine a high-risk point indicating a high incontinence risk, and notifies the user or a caregiver of the high incontinence risk at the high-risk point.

In a third aspect, a method for garment for incontinence prediction includes collecting, by one or more processors, an EIT image generated by the EIT device, a posture generated by a posture detector, and a planning activity of a user, determining, by the one or more processors, a bladder status based on the EIT image of the user, predicting, by the one or more processors, an incontinence risk based on the bladder status, the posture, and the planning activity of the user and determine a high-risk point indicating a high incontinence risk, and notifying, by the one or more processors, the user or a caregiver of the high incontinence risk at the high-risk point. The EIT device includes a plurality of electrodes configured to be worn at a lower abdomen of the user around a bladder. A first two or more electrodes are configured to apply alternating currents to a body of the user, and a second two or more electrodes record resulting potentials detected in the body of the user.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of an illustrative wearable incontinence predictor device worn on a user, according to one or more embodiments shown and described herein;

FIG. 2 schematically depicts a block schematic diagram of an electrical impedance tomography (EIT) device connected to a controller, according to one or more embodiments shown and described herein;

FIG. 3 schematically depicts a perspective view of EIT devices worn on users for two-dimensional (2D) EIT imaging and three-dimensional (3D) EIT imaging, according to one or more embodiments shown and described herein;

FIG. 4 schematically depicts a perspective view of posture detectors worn on users in front view and in back view, according to one or more embodiments shown and described herein;

FIG. 5 schematically depicts a block schematic diagram of an illustrative wearable incontinence predictor system connected with external devices, according to one or more embodiments shown and described herein;

FIG. 6 schematically depicts a block schematic diagram of a controller, according to one or more embodiments shown and described herein;

FIG. 7 schematically depicts a block schematic diagram of the training and prediction of incontinence prediction module, according to one or more embodiments shown and described herein;

FIG. 8 schematically depicts a perspective view of an illustrative a garment of wearable incontinence predictor device worn on a user, according to one or more embodiments shown and described herein;

FIG. 9 illustrates a flow diagram of an illustrative method for incontinence prediction, according to one or more embodiments shown and described herein; and

FIG. 10 illustrates a flow diagram of an illustrative method for posture detection, according to one or more embodiments shown and described herein.

### DETAILED DESCRIPTION

Embodiments described herein are directed to systems, devices, and methods that incorporate non-invasive sensors attached to a user, the sensors utilizing ultrasound technology to monitor the user's bladder status, and using monitoring data to analyze and predict incontinence risk. More specifically, the systems, devices, and methods described herein include an EIT device, a posture detector, and an incontinence event detector to establish an individualized interdependent relationship between the user's posture, bladder status, and truth of incontinence event, and predict a high risk of incontinence event based on the bladder status and the user's posture. The sensors are small and lightweight, and are designed to attach to the user's clothing or body with straps or adhesive. Upon detecting a high risk of incontinence event, the systems, devices, and methods described herein may transmit a notification to the user or a caregiver to take proper measures in coping with the risks. The systems, devices, and methods described herein may connect to external healthcare devices for other purposes, including providing notifications, triggering automations, and/or the like. The systems, devices, and methods described herein may further utilize a machine learning function that improves the detection of the bladder status and posture of the user, and the accuracy of the prediction of high-risk incontinence event of the user over time when the user continuously uses the wearable incontinence predictor.

Various embodiments of the methods, systems and devices of the wearable incontinence predictor are described in more detail herein. Whenever possible, the same reference numerals will be used throughout the drawings to refer to the same or like parts.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

EIT is an imaging technique that reconstructs images of a specific region in the human body based on the electric impedance (capacitance and resistance) of biological tissue. Biological tissue is made up of multiple cells with conductive fluid surrounded by an insulating membrane. The membrane is assimilated to the structure of a membrane capacitor (Cₘ) in parallel with a membrane resistance (Rₘ). At a low frequencies, an electric current tends to take the path of extracellular fluid because of its lower impedance. At high frequencies, the electric current is be able to pass more easily through the cell membrane and the overall conductivity of the tissues will be higher. An EIT device 101 described herein, by measuring as a function of frequency, can image complex impedance (including both capacitance and resistance) of human tissues and organs at various depth under human skins. Without limits, the EIT devices 101 disclosed herein may use different EIT systems, such as conventional EIT, dual frequency EIT, or multi-frequency EIT. The input current may be a fixed value selected from 0.2, 0.4, 0.6, 0.8, and 1.0 mA. The range of current frequency may be 1 kHz to 1 MHZ, such as 1, 2, 4, 6, 8, 10, 100, 200, 400, 600, 800, or 1000 kHz. The input AC signal may be a sinusoidal, sinusoidal, square, rectangular, sawtooth, or trapezoidal current. The obtained voltage is plotted according to the electrode array distribution to reconstruct the impedance value according to the tissues and organs underneath the human skins.

A posture detector 103 is a device that uses various sensors or cameras to monitor the position and movement of a user's body. The posture detector 103 analyzes the data collected from the sensors or cameras and determines the person's posture. The posture detector 103 herein uses, without limits, accelerometer sensors, gyroscope sensors, or unobtrusive flexible sensors. These sensors are attached to the body to detect movement and position of the user. It is noted that the posture detector 103 may include computer vision-based posture detectors using cameras to capture images of the body of the user. The posture detector 103 may use machine-learning algorithms to analyze the images to determine the posture of the users.

An incontinence event detector 102 is a device that uses various sensors to detect instances of urinary or fecal incontinence. The incontinence event detector 102 may use different sensors to identify incontinence events, such as measuring changes in skin moisture levels or detecting pressure changes in a diaper or pad. The sensors may include, without limits, moisture sensors, thermistors, or resistance meters.

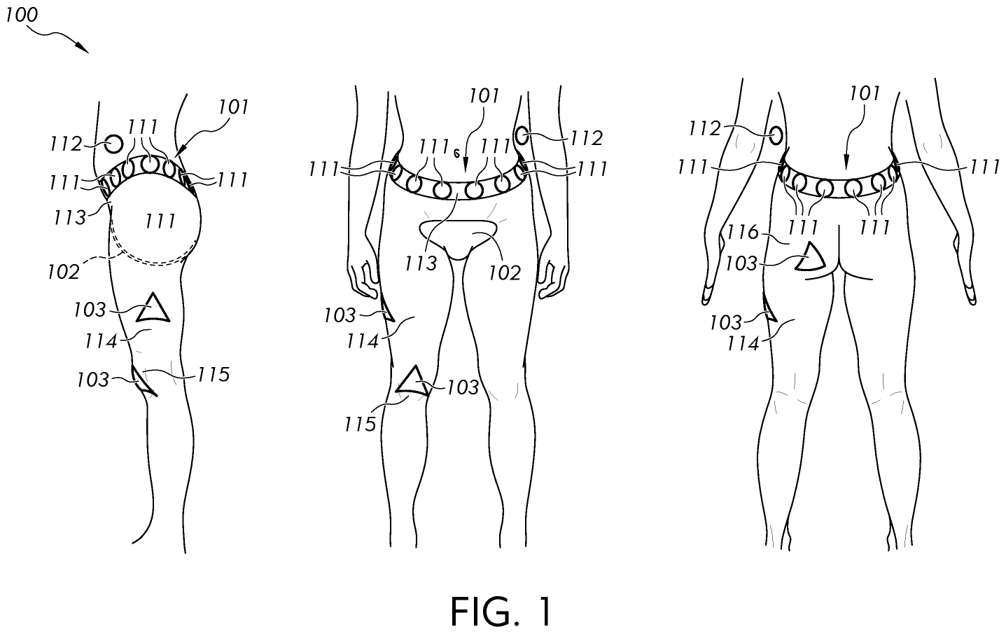

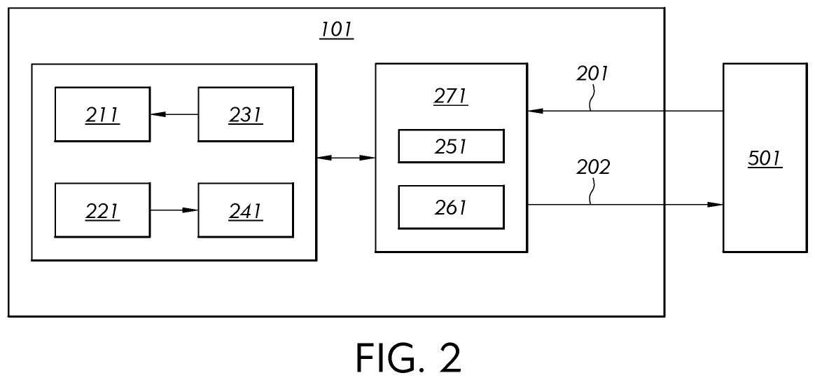

FIGS. 1-2 illustrate a wearable incontinence prediction system 100 that is wearable by a user. The wearable incontinence prediction system 100 includes an EIT device 101, one or more posture detectors 103, and a controller 501. The wearable incontinence prediction system 100 may further include an incontinence event detector 102. As illustrated in FIG. 1, the user wears a EIT device 101 at the lower abdomen 113 of the user around the bladder. The EIT device 101 includes a plurality of electrodes 111 configured to be worn at the lower abdomen 113 of the user around the bladder. For instance, as in FIG. 1, the electrodes 111 are arranged in a belt shape (distributed in a circle or oval shape, as if worn on a person using a belt or a waistband), evenly attached to the user around the user's lower abdomen 113. Among the electrodes 111, a first two or more electrodes 211 are configured to apply alternating currents generated using a programmable current simulator 231 (an AC signal generator, including parts like an oscillator to generate AC signals) to a body of the user, and a second two or more electrodes 221 record resulting potentials detected in the body of the user. As in FIG. 1, the user may wear one or more posture detectors attached to or around the user's hips 116, buttocks, thighs 114, or knees 115. In some embodiments, the posture detectors 103 may be attached to or embedded in a cloth, a pad, or an accessory, where the posture detectors 103 do not directly contact the user. The user may wear an incontinence event detector 102 attached to or around the user's crotch, groin, buttocks, or lower abdomen 113. While FIG. 1 shows possible locations where the EIT device 101, the incontinence event detector 102, and the posture detector 103 may be worn, it should be understood that a user may wear these devices and detectors anywhere on the body as long as the devices and detectors serve their intended functions outlined in this disclosure.

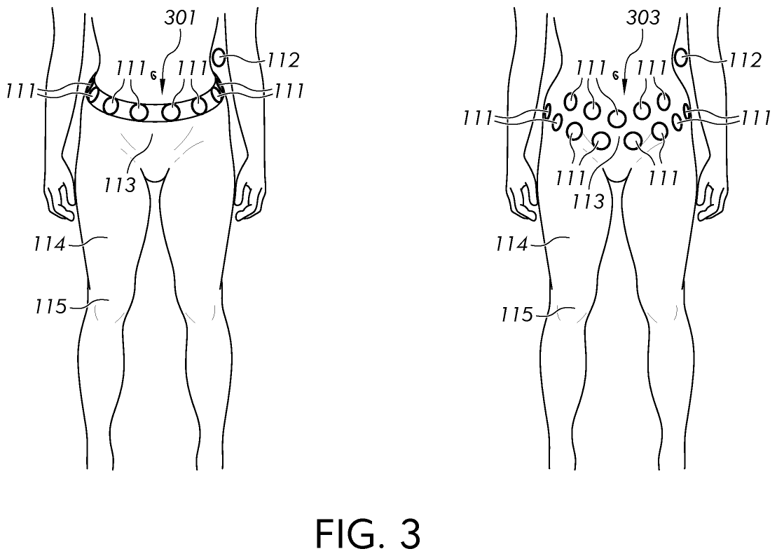

The EIT device 101 measures the impedance signals based on the potential signals collected by the second set of electrodes 221. The measurements are carried out using a plurality number of electrodes 111. The electrode shape may be circular, square, rectangular, or oval. The electrodes 111 may be of a biocompatible material, such as gold, silver, platinum, carbon, conductive polymers, or hydrogels. The choice of the shape and size may be dependent on the applied voltage and current density. For example, a high current density may be associated with a large electrode area. In some embodiments, the number of electrodes is 4, 6, 8, 10, 12, 14, 16, 18, or 20 electrodes. In some other embodiments, the number of electrodes is 16, 32, 64, 128, 256, or 512 electrodes. The electrode number may be dependent on the EIT imaging requirements. For example, for a low-resolution or two-dimensional (2D) EIT imaging (band shape electrodes 301 in FIG. 3), the electrode number may be below 20; for a high-resolution or three-dimensional (3D) EIT imaging (multiple-array structure electrodes 303 in FIG. 3), the electrode number may be above 64 (e.g., more than 64 electrodes). The electrodes 111 may be supported on a belt, a band, or a rigid plane, from which each electrode protrudes. The EIT device may include a programmable current simulator 231 as an alternating current (AC) source. The EIT device may further have an output multiplexer coupled to the electrodes 111 at one end and coupled to the programmable current simulator 231 on the other end. An input multiplexer may connect at least one of the second set of electrodes 221 to a voltage sensor 241, such as a potential difference measuring unit. The voltage sensor 241 is coupled to the controller 501 through an electrode switching circuit 271, which includes an AC/DC converter 251 and an analog-to-digital converter 261.

Referring to FIGS. 1-2, a controller 501 is communicatively coupled to the EIT device 101 and the posture detectors 103. The controller 501 is configured to collect EIT signals and EIT images generated by the EIT device, postures generated by the posture detector, and one or more planning activities of the user. The controller 501 may be further configured to collect incontinence information generated by the incontinence event detector 102.

FIG. 2 illustrates the EIT device communicatively coupled to a controller 501 (e.g., via a wired or wireless connection). The wireless connection may be achieved using a wireless communication protocol such as Bluetooth, Wi-Fi, Zigbee, Z-Wave, or the like. The EIT device 101 includes a plurality of electrodes 111, with the first two or more electrodes 211 (hereinafter “first set of electrodes” or “first two or more electrodes”), and the second two or more electrodes 221 (hereinafter “second set of electrodes” or “second two or more electrodes”). The EIT device further includes a programmable current simulator 231 coupled to the first set electrodes 211 and a voltage sensor 241 coupled to the second set of electrodes 221.

The electrodes 111 are attached to tissue of the user (e.g., skin) with a low-contact impedance that may overcome the negative influence of certain tissue characteristics, such as skin hydration and other wet conditions. The programmable current simulator 231 may include an oscillator. The oscillator may be a LC oscillator, an Armstrong oscillator, a Hartley oscillator, a Colpitts oscillator, or a Crystal oscillator. The programmable current simulator 231 may be coupled to the electrode switching circuit 271 and may be further controlled by the controller 501.

The second set of electrodes 221 is coupled to the voltage sensor 241 through the input multiplexer. The EIT device may include a non-contact electrode 112, which does not directly contact the skin of the user or is not in a low-contact impedance.

The EIT device may include an AC/DC converter 251 and an analog-to-digital converter 261 to generate real parts and image parts of the impedance.

FIG. 3 illustrates the belt shape electrodes 301 for 2D EIT imaging on the left and the multiple-array structure electrodes 303 for 3D EIT imaging on the right. For the belt shape electrodes 301, the electrodes 111 may be arranged in a singular planar array of electrodes such that the collected impedance data 202 represents the potential response within that plane. For the multiple-array structure electrodes 303, the electrodes 111 may be arranged in a matrix in various shapes, such as square, triangle, trapezoidal, or the like.

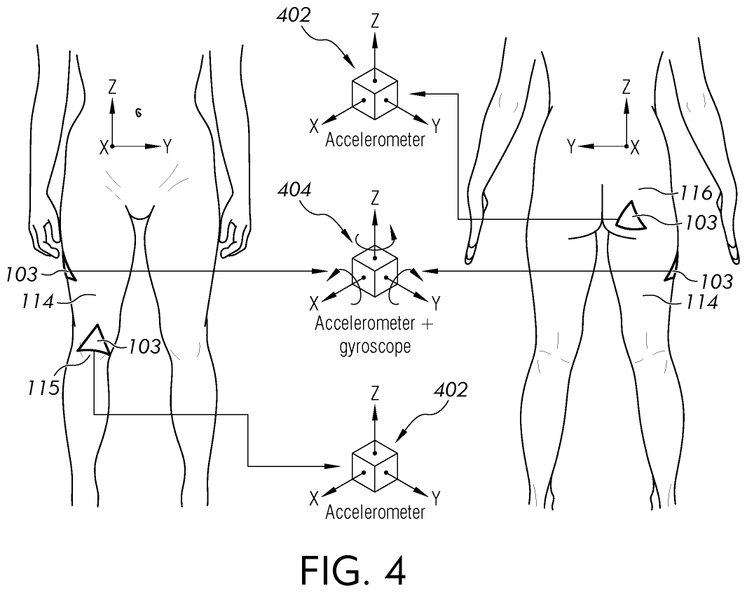

FIG. 4 depicts a perspective view of posture detectors 103 worn on a user. The posture detectors 103 may be worn by the user on clothes, such as tight-fitting leggings or shorts, with the sensors attached to the clothes. Alternatively, the sensors may be attached directly to the skin of the user with adhesive patches or straps. In embodiments, as shown in FIG. 4, the user may wear one or more posture detectors 103 attached to or around the user's hips 116, buttocks, thighs 114, or knees 115. The posture detector 103 worn around the knees 115 may be positioned just above the kneecap, while posture detector 103 worn around the thigh 114 may be located midway up the thigh 114. The posture detector 103 worn around hip 116 may be attached to the user's waistband or belt, positioned over the hip joint. The posture detection sensor may be an accelerometer, a gyroscope, or an unobtrusive flexible sensor to generate posture data.

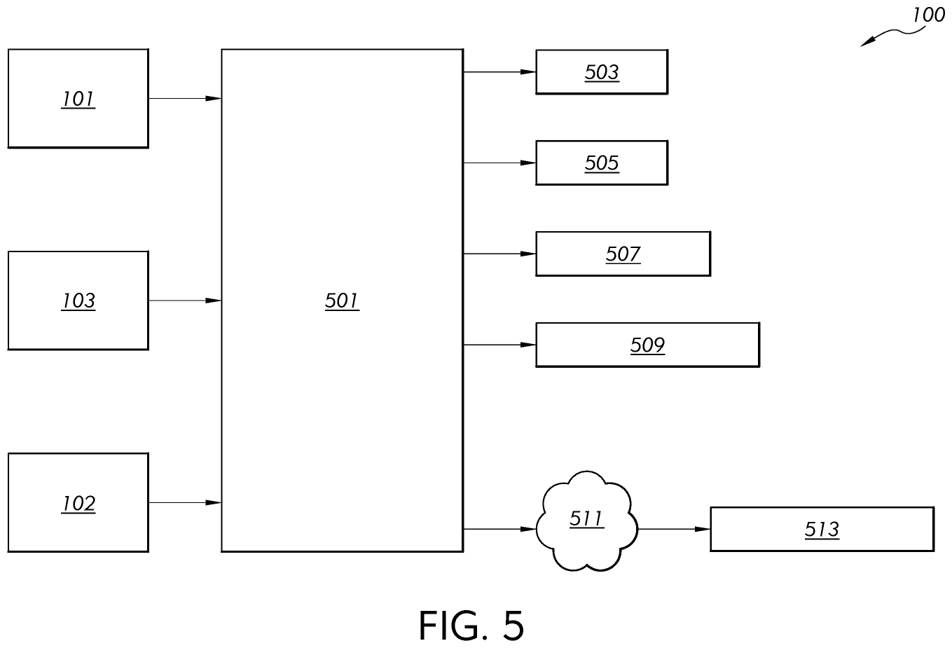

FIG. 5 depicts a block schematic diagram of an illustrative wearable incontinence prediction system 100 connected with external devices. Particularly, the EIT device 101, posture detector 103, and incontinence event detector 102 are coupled to controller 501 through a wired or wireless connection. The controller 501 may further connect with external devices, such as dashboard 503, nurse system 505, infusion pump 507, a notification receiver 513, or a female catheter 509. The controller 501 may connect with external devices via cables or wirelessly, such as a network 511. The notification receiver 513 may be a user's or a caregiver's mobile device, smartphone, smartwatch, or the like.

In embodiments, the network 511 may include, for example, one or more computer networks (e.g., a personal area network, a local area network, grid computing network, wide area network, etc.), cellular networks, satellite networks and/or a global positioning system and combinations thereof. Suitable local area networks may include wired Ethernet and/or wireless technologies such as, for example, wireless fidelity (Wi-Fi). Suitable personal area networks may include wireless technologies such as, for example, IrDA, Bluetooth, Wireless USB, Z-Wave, ZigBee, and/or other near field communication protocols. Suitable personal area networks may similarly include wired computer buses such as, for example, USB and Fire Wire. Suitable cellular networks include, but are not limited to, technologies such as LTE, WiMAX, UMTS, CDMA, and GSM.

In embodiments, the network 511 may include, for example, one or more computer networks (e.g., a personal area network, a local area network, grid computing network, wide area network, etc.), cellular networks, satellite networks and/or a global positioning system and combinations thereof. Accordingly, the controller 501, the EIT device 101, the posture detector 103, the incontinence event detector 102, the external devices, and the notification receiver 513 can be communicatively coupled to the network 110 and/or one another via wires, via a wide area network, via a local area network, via a personal area network, via a cellular network, via a satellite network, or the like.

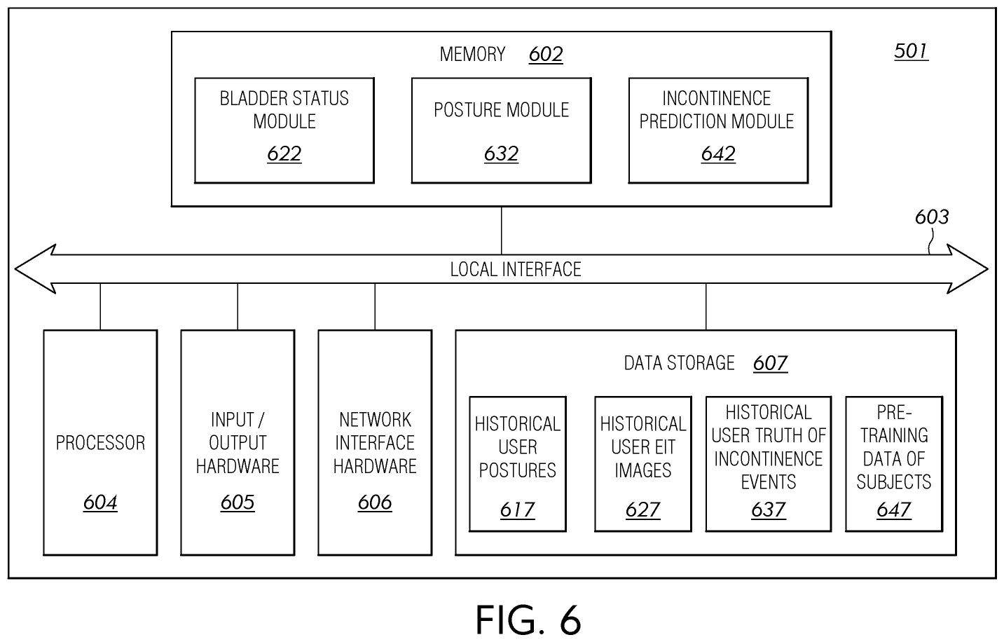

FIG. 6 depicts a block schematic diagram of the controller 501. The wearable incontinence prediction system 100 may include a bladder status module, a posture module 632, an incontinence prediction module 642.

The controller 501 may include a computing device, which may be any device or combination of components including a processor 604 and a memory 602, such as a non-transitory computer readable memory. The processor 604 may be any device capable of executing the machine-readable instruction set stored in the non-transitory computer readable memory. Accordingly, the processor 604 may be an electric controller, an integrated circuit, a microchip, a computer, or any other computing device. The processor 604 may include any processing component(s) configured to receive and execute programming instructions (such as from the data storage component 607 and/or the memory component 602). The instructions may be in the form of a machine-readable instruction set stored in the data storage component 607 and/or the memory component 602. The processor 604 is communicatively coupled to the other components of the computing device by the local interface 603. Accordingly, the local interface 603 may communicatively couple any number of processors 604 with one another, and allow the components coupled to the local interface 603 to operate in a distributed computing environment. The local interface 603 may be implemented as a bus or other interface to facilitate communication among the components of the computing device. In some embodiments, each of the components may operate as a node that may send and/or receive data. While the embodiment depicted in FIG. 6 includes a single processor 604, other embodiments may include more than one processor 604.

The memory 602 (e.g., a non-transitory computer readable memory component) may include RAM, ROM, flash memories, hard drives, or any non-transitory memory device capable of storing machine-readable instructions such that the machine-readable instructions can be accessed and executed by the processor 604. The machine-readable instruction set may include logic or algorithm(s) written in any programming language of any generation (e.g., 1GL, 2GL, 3GL, 4GL, or 5GL) such as, for example, machine language that may be directly executed by the processor 604, or assembly language, object-oriented programming (OOP), scripting languages, microcode, etc., that may be compiled or assembled into machine readable instructions and stored in the memory 602. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 602 may be a machine-readable memory (which may also be referred to as a non-transitory processor readable memory or medium) that stores instructions which, when executed by the processor 604, causes the processor 604 to perform a method or control scheme as described herein. While the embodiment depicted in FIG. 6 includes a single non-transitory computer readable memory 602, other embodiments may include more than one memory module.

The input/output hardware 605 may include a monitor, keyboard, mouse, printer, camera, microphone, speaker, and/or other device for receiving, sending, and/or presenting data. The network interface hardware 606 may include any wired or wireless networking hardware, such as a modem, LAN port, Wi-Fi card, WiMax card, mobile communications hardware, and/or other hardware for communicating with other networks and/or devices.

The data storage component 607 stores historical user postures 617, historical user EIT images 627, historical user truth of incontinence events 637, and pre-training data of subjects 647. It should be understood that the data storage component 607 may reside local to and/or remote from the computing device and may be configured to store one or more pieces of data for access by the computing device and/or other components.

The memory component 602 may include a bladder status module 622, a posture module 632, and an incontinence prediction module 642. Additionally, the memory 602 may store data generated in the bladder status module 622, the posture module 632, and the incontinence prediction module 642, such as a neural network model therein. The bladder status module 622, the posture module 632, and the incontinence prediction module 642 may further include one or more neural network models having a machine learning function. For example, the incontinence prediction module 642 may include a prediction artificial neural network (ANN) model 711.

In embodiments, the one or more neural networks including the incontinence prediction module 642 may be trained and provided machine learning capabilities via a neural network as described herein. By way of example, and not as a limitation, the neural network may utilize one or more ANNs. In ANNs, connections between nodes may form a directed acyclic graph (DAG). ANNs may include node inputs, one or more hidden activation layers, and node outputs, and may be utilized with activation functions in the one or more hidden activation layers such as a linear function, a step function, logistic (sigmoid) function, a tanh function, a rectified linear unit (ReLu) function, or combinations thereof. ANNs are trained by applying such activation functions to training data sets to determine an optimized solution from adjustable weights and biases applied to nodes within the hidden activation layers to generate one or more outputs as the optimized solution with a minimized error. In machine learning applications, new inputs may be provided (such as the generated one or more outputs) to the ANN model as training data to continue to improve accuracy and minimize error of the ANN model. The one or more ANN models may utilize one to one, one to many, many to one, and/or many to many (e.g., sequence to sequence) sequence modeling. The one or more ANN models may employ a combination of artificial intelligence techniques, such as, but not limited to, Deep Learning, Random Forest Classifiers, Feature extraction from audio, images, clustering algorithms, or combinations thereof.

In some embodiments, a convolutional neural network (CNN) may be utilized. For example, a convolutional neural network (CNN) may be used as an ANN that, in a field of machine learning, for example, is a class of deep, feed-forward ANNs applied for audio-visual analysis of the captured disturbances. CNNs may be shift or space invariant and utilize shared-weight architecture and translation.

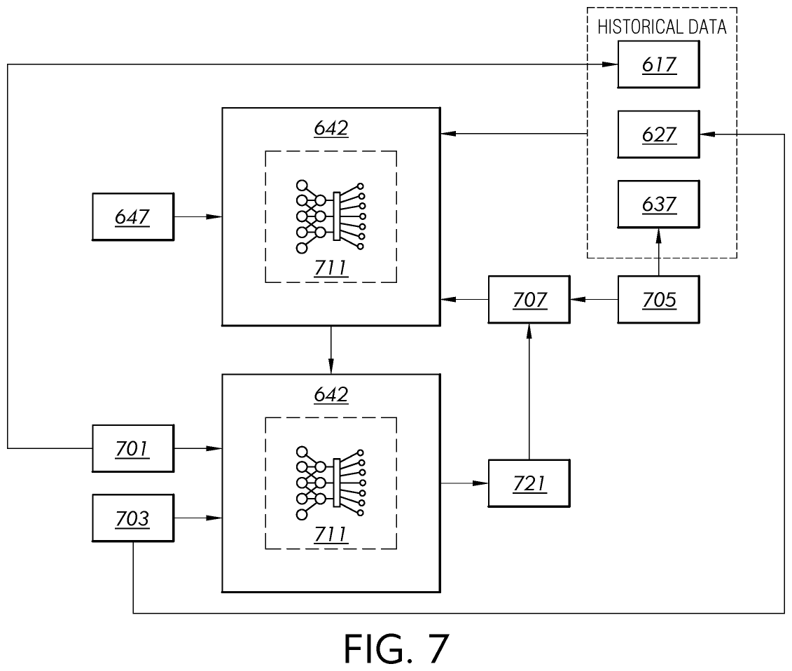

FIG. 7 depicts a block schematic diagram of the training and prediction of incontinence prediction module. The controller 501 has an incontinence prediction module 642. The incontinence prediction module 642 receives real-time posture data 701 and the real-time EIT images 703. Accordingly, the incontinence prediction module 642 may predict the risk of incontinence by considering factors including the real-time postures 701 and real-time EIT images 703 reflecting the bladder status of the user.

The incontinence prediction module 642 may further include a prediction artificial neural network (ANN) 711. The prediction ANN 711 may be pretrained with pre-training posture-status data including posture data indicating subject postures, EIT image data indicating subject bladder statuses, and incontinence data indicating subject incontinence events. Upon the incontinence prediction module 642 is fully trained, the trained incontinence prediction module 642 is used to predict the risk of incontinence of the user based on input data such as real-time postures 701, real-time EIT images 703, and planning activity.

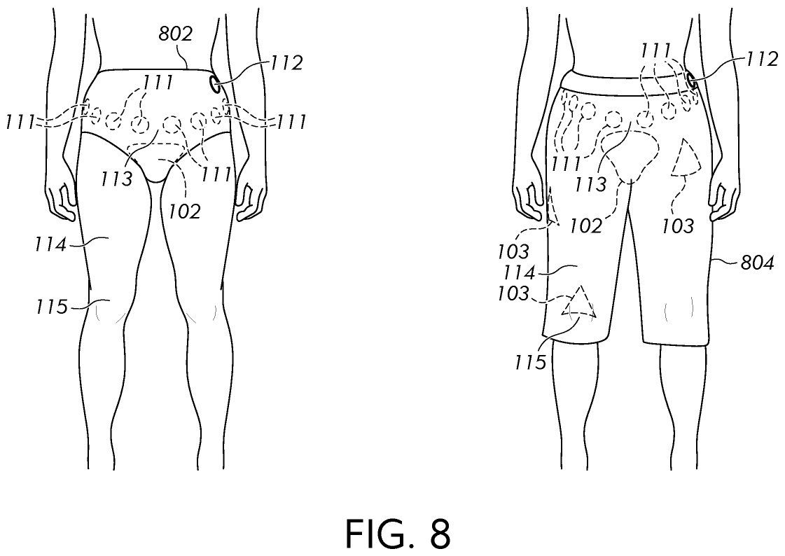

In embodiments, the trained incontinence prediction module 642 may be continuously updated by validation 707 of the predicted incontinence risk with collected data of the user postures, the EIT images, and the truth of user incontinence events. The output predicted high incontinence risk 721 (i.e. predicted incontinence risk) generated by the trained Incontinence prediction module 642 may be validated by a real-time truth of incontinence event 705. The validation 707 results may be fed to the prediction ANN 711 for retraining. The real-time truth of incontinence event may be generated by an incontinence event detector 102 (e.g. as illustrated in FIGS. 1 and 8), which may be included in the wearable incontinence prediction system 100. The real-time truth of incontinence event 705 may be included in the pool historical user truth of incontinence events 637 after the occurrence of the validation 707. In yet some embodiments, the incontinence prediction module 642 may be validated with historical data, which includes historical user posture data 617, historical user EIT images 627, and historical user truth of incontinence events 637 generated since the initial usage of the EIT device by the user.

Referring to FIG. 8, an example garment for incontinence prediction is illustrated. In embodiments, the wearable incontinence prediction system 100 including a garment may include a garment body formed of a flexible fabric, wherein the garment body is configured to be worn on a lower torso of a user. The electrical impedance tomography (EIT) device 101 may be configured to be attached on the garment. For example, the electrodes 111 may be arranged at discrete locations on the garment body, wherein each electrode is positioned on an inner surface of the garment body and is configured to contact at a lower abdomen 113 of the user around the bladder. The posture detector 103 may be arranged at an outer or inner surface of the garment body. The garment may further include an incontinence event detector 102 arranged at an inner surface of the garment body to identify truth of an incontinence event. In embodiments, the garment may include a pair of undergarments, where the pair of undergarments is a pair of boxers 804, a pair of briefs 802, a pair of boxer briefs, a pair of trunks, a pair of midway briefs, or a pair of thermal underwear.

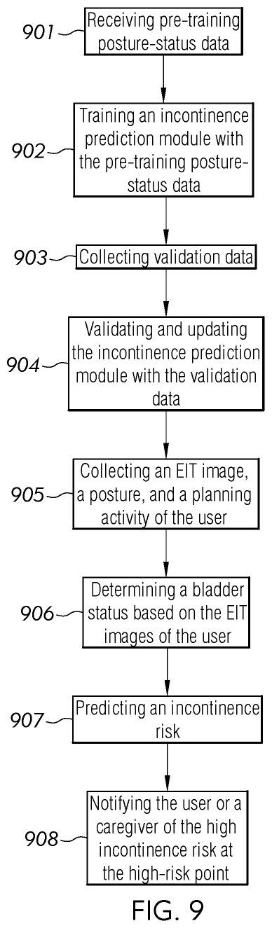

Referring to FIG. 9, an example flow diagram of an illustrative method for incontinence prediction is illustrated. The method can be used by a system or apparatus, such as the wearable incontinence prediction system 100 described herein.

At block 901, the method may include a step of receiving pre-training posture-status data including posture data indicating subject postures, EIT image data indicating subject bladder statuses, and incontinence data indicating subject incontinence events. At block 902, the method may include a step of training an incontinence prediction module 642 (e.g., as illustrated in FIG. 6) with the pre-training posture-status data by modeling dependence between the subject postures, the subject bladder statuses, and truth of the subject incontinence events.

Referring to FIG. 7, the prediction ANN 711 of the incontinence prediction module 642 may be pretrained with pre-training posture-status data including posture data indicating subject postures, EIT image data indicating subject bladder statuses, and incontinence data indicating subject incontinence events. The pre-training posture-status data are collected from one or more subjects who have symptoms of incontinence. The pre-training posture-status data may reflect to a specific type of incontinence, such as stress incontinence, urge incontinence, overflow continence, functional incontinence, or mixed incontinence. The user may select a set of pre-training posture-status data that matches to the symptoms of the user to pretrain the prediction ANN 711 or the incontinence prediction module 642. The pre-training may be conducted by modeling dependence between the subject postures, the subject bladder statuses, and truth of the subject incontinence events. Upon the incontinence prediction module 642 is fully trained, the trained incontinence prediction module 642 is used to predict the risk of incontinence of the user based on input data such as real-time postures 701, real-time EIT images 703, and planning activity. During the training, the incontinence prediction module 642 may weigh the validation data more than the pre-training posture-status data.

Referring back to FIG. 9, at block 903, the method may include a step of collecting validation data including historical data including user postures, EIT images, and truth of user incontinence events generated since an initial usage of the EIT device 101 (e.g., as illustrated in FIG. 1) by a user. At block 904, the method may include a step of validating and updating the incontinence prediction module 642 (e.g., as illustrated in FIG. 6) with the validation data. As illustrated in FIGS. 6-7, the incontinence prediction module 642 may be validated with historical data, which includes historical user posture data 617, historical user EIT images 627, and historical user truth of incontinence events 637 generated since the initial usage of the EIT device by the user.

Further, the trained incontinence prediction module 642 may be continuously updated by validation 707 of the predicted incontinence risk with collected data of the user postures, the EIT images, and the truth of user incontinence events. An incontinence event detector 102 may generate the truth of user incontinence event. In embodiments, the wearable incontinence prediction system 100 may include an incontinence event detector 102, which may be attached to a diaper or pad work and detect changes in moisture levels or temperature caused by urine leakage. The incontinence event detector 102 may be a moisture sensor, a thermistor, or a resistance meter. The moisture sensor may be triggered to send a positive signal when urine comes into contact with the moisture sensor as the urine changes the electrical conductivity between two conductive strips. The thermistor may detect a change in temperature, indicating the presence of urine when the thermistor measures a change in temperature caused by urine. The resistance meter may send a positive signal when urine contact with the sensor and cause a change in the electrical resistance of the sensor. The controller 501 upon receiving a positive signal may determine whether an incontinence event happens and how severity is the urine leaking. In some embodiments, the wearable incontinence prediction system 100 may include a user interface to allow a user to input the occurrence of an incontinence event. The generation of user postures and EIT images are described in detail below in blocks 905 and 906.

Referring again to FIG. 9, at block 905, the method may include a step of collecting an EIT image, a posture, and a planning activity of the user. At block 906, the method may include a step of determining a bladder status based on the EIT images of the user.

Referring to FIGS. 1-2, the EIT images are generated using the EIT device 101. The EIT device 101 measures the impedance signals based on the potential signals collected by the second set of electrodes 221. The measurements are carried out using a plurality number of electrodes 111. The EIT device 101 includes a plurality of electrodes 111, with the first two or more electrodes 211 configured to apply alternating currents to the body of the user, and the second two or more electrodes 221 configured to record resulting potentials detected in the body of the user. The programmable current simulator 231 may supply AC to the first set of electrodes 211 through the connection of the output multiplexer. It has to be noted that electrodes 111 used as first set of electrodes 211 in one measurement may be later used as second set of electrodes 221 to receive induced potential in another measurement, for different reasons to incur a different current distribution or to reconstruct a different EIT image.

The programmable current simulator 231 coupled to the first set electrodes 211 may apply the alternating currents on the first set electrodes 211. The voltage sensor 241 coupled to the second set of electrodes 221 may receive potential signals collected by the second set of electrodes 221. The current applied generated from the programmable current simulator 231 may be small signals at a low voltage and a low current density. The programmable current simulator 231 may include an oscillator to general AC signals, such as sinusoidal, square, rectangular, sawtooth, and trapezoidal waveshapes. The oscillator may be a LC oscillator, an Armstrong oscillator, a Hartley oscillator, a Colpitts oscillator, or a Crystal oscillator. In embodiments, the applied current is 0.2, 0.4, 0.6, 0.8, or 1.0 mA. The current frequency is at 1, 2, 4, 6, 8, or 10 kHz. The current are applied to the first set of electrodes 211, which connect to the programmable current simulator. The programmable current simulator 231 may be coupled to the electrode switching circuit 271 and may be further controlled by the controller 501. In one embodiment, the programmable current simulator 231 may have preset programs to generate current. In another embodiment, the programmable current simulator 231 generates current under the control of the controller 501. In embodiments, the electrodes 111 may be activated one electrode or a pair of electrodes at a time by the programmable current simulator 231. The voltage sensor 241 measures the difference between the second set of electrodes 221 and the non-contact electrode 112.

In embodiments, the AC/DC converter 251 may convert the detected AC voltage into a DC voltage before analogue-to-digital conversion. The DC signals generated by the AC/DC converter 251 may be then fed into the analog-to-digital converter 261. The analog-to-digital converter 261 may sample the DC signals at a high frequency and may convert each sample into a digital value, which may be further used to calculate the resistance of the user body at different depths for creating EIT images.

Referring to FIGS. 2-3, the controller 501 as illustrated in FIG. 2 may receive the electrode arrangement and contact impedance information for the purpose of image reconstruction from the measurements. A mathematical algorithm may be used to determine a sufficiently low-impedance contact between the electrode and user's skin by measuring the internal conductivity distribution of the electrodes 111 that would produce the measured voltage readings. In some embodiments, the wearable incontinence prediction system 100 may assign a threshold voltage value to exclude high-impedance contact between the electrode and the user's skin such that any voltage measured below the threshold voltage is excluded for EIT image reconstruction. The controller 501 may also tune the input AC current accordingly to arouse a higher response voltage to raise the threshold voltage. The mathematical algorithm used may depend on the geometry of the electrode arrays, such as belt shape or multiple-array structure.

The controller 501, including a processor, uses the sensor signals and the applied currents to determine potential difference measurements. The controller 501 sends commands 201 to and receives impedance data 202 from the EIT device 101. The commands 201 may include instructions for applying AC or DC currents to the user, detecting voltage/potential, converting measured voltage/potential into real and image parts, or amplitude and phase, and the like.

After the controller 501 collects current and voltage signals and converts the signals into impedance signals, the controller 501 may then convert the signals into an EIT image. For that purpose, the controller 501 may determine a bladder impedance corresponding to liquid volume in the bladder and a background impedance corresponding to background tissues. The background impedance may correspond to the background tissues including pelvic internal organs, muscle, and fat of the user. The controller 501 may conduct a dynamic analysis based on the background impedance, which is constant, and the bladder impedance changes, which accord liquid volume change in the bladder. After the controller 501 reconstructs an EIT image, the controller 501 may determine the bladder volume of the user based on the correlation between the bladder impedance and the liquid volume in the bladder. Such correlation between the bladder impedance and the liquid volume may be determined by the wearable incontinence prediction system 100 through a machine learning function. In embodiments, the bladder impedance change may be linear with the liquid volume in the bladder.

Based on the EIT image and the determined liquid volume in the bladder, the wearable incontinence prediction system 100 may further determine a bladder status based on the EIT images. The bladder status may include a non-full status and a full status. The bladder status may further include the non-full status including an empty status, a half-full status, and a three-fourths full status. In embodiments, the full status is based on a maximum bladder tolerance of the user. The maximum bladder tolerance of the user may correspond to a largest liquid volume in the user bladder based on collected historical EIT images since an initial usage of the EIT device by the user.

Referring to FIG. 4, a posture may be detected using a posture detector 103. The posture detectors 103 may be small and unobtrusive, designed to track the user's movement patterns and provide data on their gait, balance, and posture. The posture detectors 103 may generate posture data and transmit the posture data through a cable or wirelessly to the controller 501 for analysis and interpretation.

As shown in FIGS. 4-5, accelerometers worn by the user on the right hip 116, right thigh 114, and right knee 115 may detect acceleration of the body parts in any direction in the Cartesian coordinates (i.e. a vector (x, y, z) representing the amplitude and direction in the three-dimensional coordinates), and a gyroscope may detect the rotation movement of the body parts in any circular direction in the Cartesian coordinates. An accelerometer thus may detect three-dimensional forces/accelerations 402 and a gyroscope may detect three-dimensional torques. An accelerometer and a gyroscope together thus may monitor a six-dimensional movement 404 of a body part. By wearing multiple accelerometers and gyroscopes on different body parts of the user, the wearable incontinence prediction system 100 may collect acceleration and rotation data in determining the movement, orientation, and steadiness of these body parts. Particularly, the accelerometer may detect the direction in which the sensor is oriented relative to the Earth's gravity, such as being held upright or tilted, and calculate the speed at which the sensor is moving. Thus, an accelerometer may generate signals depicting the relevant orientation and movements of the position attached. A gyroscope, on the other hand, may measure the speed of rotation of the sensor, which can be used to track the movement of the user's thighs 114 or limbs. The gyroscope may determine whether the body part of the user is moving in a particular direction, such as turning or twisting. Further, the gyroscope may detect changes in the stability of that body part of the user, and measure the speed of rotation of that body part of the user. As such, the posture module 632 of the controller 501 may use the detected posture data to determine the posture of the user, such as a sitting posture, a standing posture, a flat laying posture, an inclined laying posture, or a side laying posture.

Further, the posture may be an unobtrusive flexible sensor, such as a piezoelectric flexible sensor. The unobtrusive flexible sensor may generate electric signals as posture data based on the shape change of piezoelectric materials. For example, the unobtrusive flexible sensor may generate opposite pulse currents in a bent state compared with a release state. Multiple unobtrusive flexible sensors may be attached to the clothing worn by the user or placed on different parts of the body conforming to the shape of the body. The collected pulse current signals may be send to the controller 501 to determine movements of various body parts that involve bending and releasing and determine the activity and posture of the user, such as sitting, walking, running, and jumping. Moreover, the electric signals generated by multiple unobtrusive flexible sensors may be used to depict the bending and releasing states of different body parts, where the posture module 632 (e.g. as illustrated in FIG. 6) of the controller 501 may use the combination of the states of these body parts to detect the posture of the user, such as standing, flat laying, inclined laying, and a side laying. The unobtrusive flexible sensor may be further used to provide validation of the user's input of planning activities and measure the intensity of such planning activities to create linkages between the activities and risk of incontinence.

In embodiments, the posture module 632 (e.g., as illustrated in FIG. 6) may include a posture classification to determine the posture of the user. Particularly, after the controller 501 collecting posture data from the posture detector, such as acceleration, torque, bending, releasing data of body parts like hip 116, knee 115, thighs 114, the posture module 632 (e.g., as illustrated in FIG. 6) may first use signal processing techniques to filter and analyze the data. The posture module 632 (e.g., as illustrated in FIG. 6) may then use the processed data to extract features related to the posture of the user, such as joint angles and body position. After the features have been extracted, the posture module 632 (e.g., as illustrated in FIG. 6) may conduct a posture classification using a machine learning algorithms to classify the user's posture. The machine learning algorithm may be trained using a dataset of labeled postures, where the posture detector 103 has collected data from users in different postures. The algorithm may learn to recognize patterns in the data that correspond to different postures.

In embodiments, the wearable incontinence prediction system 100 may have a function to allow the user to provide posture feedback. After the user's posture has been classified, the controller 501 may provide feedback to the user for further validation. For example, if the user is slouching, the controller 501 can provide a visual signal that prompts the user to sit up straight. As such, the controller 501 may update to the user's posture over time when the posture detector 103 may continuously collect data on the user's posture and adjust the posture module and the classification algorithm accordingly. This can improve the accuracy of the posture detection wearable incontinence prediction system 100 over time.

Further, the wearable incontinence prediction system 100 may have a function to allow the user to input the user's planning activity. Different activities may apply different levels of pressure on the bladder, causing urine leakage. For example, hunching, moderate or deep coughing, sneezing, laughing, exercising, or lifting may increase the risk of incontinence at a lower level of bladder status. Accordingly, the incontinence prediction module 642 may predict the risk of incontinence by considering factors including planning activity and real-time EIT images 703 reflecting the bladder status of the user.

Referring again to FIG. 9, at block 907, the method may include a step of predicting an incontinence risk based on the bladder status, the posture, and the planning activity of the user and determining a high-risk point indicating a high incontinence risk. Referring to FIGS. 5-7, the controller 501 may have an incontinence prediction module 642. The incontinence prediction module 642 receives real-time posture data 701 generated by the posture module 632 based on the posture data generated by the posture detector 103 and bladder status generated by the bladder status module 622 based on the real-time EIT images 703, and use these data to predict the risk of incontinence. For example, a bladder status of full or three-fourths full may suggest a high liquid volume in the bladder in corresponding to a high risk of incontinence event of the user. The incontinence prediction module 642 may evaluate the incontinence risk of the user at a same liquid volume in the bladder by considering other factors. For example, the incontinence prediction module 642 may tend to generate a high risk of innocence event for certain postures and activities of the user, even the bladder status is half-full or nearly empty. For example, the user in an upright position (such as sitting or standing) may have a higher risk of incontinence than the user in the position lying in a bed. Accordingly, the incontinence prediction module 642 may predict the risk of incontinence by considering factors including the real-time postures 701 and real-time EIT images 703 reflecting the bladder status of the user.

The wearable incontinence prediction system 100 may include a bladder status module 622 to determine a bladder status based on the EIT image of the user, a posture module 632 to determine a posture of the user, an incontinence prediction module 642 to predict an incontinence risk based on the bladder status, the posture, and the planning activity of the user. The incontinence prediction module 642 may further determine a high-risk point indicating a high incontinence risk.

Referring again to FIG. 9, at block 908, the method may include a step of notifying the user or a caregiver of the high incontinence risk at the high-risk point. Referring to FIG. 5, the notification receiver 513 may include a graphic user interface such that when the wearable incontinence prediction system 100 determines a high-risk point indicating a high incontinence risk, the user or the caregiver may receive a notification. The controller 501 may further detect dehydration of the user based on the bladder status and transmits an alarm to the dashboard 503 or the nurse system 505. The controller 501 may further transmit the bladder status and the incontinence risk to an external infusion pump 507, where the external infusion pump 507 may adjust flow rates of fluids and nutrients to the user based on the bladder status or the incontinence risk. The controller 501 may further transmit the bladder status and the incontinence risk to an external female catheter 509, where the external female catheter 509 may adjust the vacuum pump output based on the bladder status or the incontinence risk.

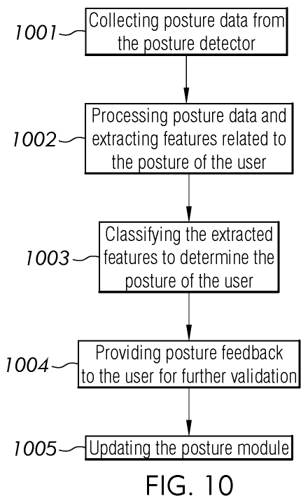

Referring to FIG. 10, an example flow diagram of an illustrative method for posture detection is illustrated. The method can be used by a system or apparatus, such as the incontinence prediction wearable incontinence prediction system 100 described herein. The steps allows the controller 501 (e.g., as illustrated in FIGS. 4-5) to determine the user's posture using a posture detector 103 (e.g., as illustrated in FIGS. 4-5) and provide feedback to the user to validate the posture of the user.

At block 1001, the method may include a step of collecting posture data from the posture detector 103. The controller 501 (e.g., as illustrated in FIGS. 4-5) may collect posture data from the posture detector 103 (e.g., as illustrated in FIGS. 4-5), which collect data on the movement and position of the user's body. The posture data may include acceleration, torque, bending, releasing data of body parts like hip 116 (e.g., as illustrated in FIGS. 4-5), knee 115 (e.g., as illustrated in FIGS. 4-5), thighs 114 (e.g., as illustrated in FIGS. 4-5).

At block 1002, the method may include a step of processing posture data. The posture module 632 (e.g., as illustrated in FIG. 6) may process the data collected from the posture detector 103 (e.g., as illustrated in FIGS. 4-5) by using signal processing techniques to filter and analyze the data. The posture module 632 (e.g., as illustrated in FIG. 6) may then use the processed data to extract features related to the posture of the user, such as joint angles and body position.

At block 1003, the method may include a step of posture classification. After the features have been extracted, the posture module 632 (e.g., as illustrated in FIG. 6) may use machine learning algorithms to classify the user's posture. The machine learning algorithm may be trained using a dataset of labeled postures, where the posture detector 103 has collected data from users in different postures. The algorithm may learn to recognize patterns in the data that correspond to different postures.

At block 1004, the method may include a step of posture feedback. After the user's posture has been classified, the controller 501 (e.g., as illustrated in FIG. 6) may provide feedback to the user for further validation. For example, if the user is slouching, the controller 501 (e.g., as illustrated in FIG. 6) can provide a visual signal that prompts the user to sit up straight.

At block 1005, the method may include a step of user updating. Finally, the controller 501 (e.g. as illustrated in FIGS. 4-5) may update to the user's posture over time. The posture detector 103 (e.g. as illustrated in FIGS. 4-5) may continuously collect data on the user's posture over time and adjust the posture module 632 (e.g. as illustrated in FIG. 6) and the classification algorithm accordingly. This can improve the accuracy of the posture detection wearable incontinence prediction system 100 (e.g. as illustrated in FIGS. 4-5) over time.

It should now be understood that embodiments described herein are directed to systems and methods for incontinence prediction. The system described herein may include an EIT device, a posture detector, a controller. The system may collect an EIT image generated by the EIT device, a posture generated by the posture detector, and a planning activity of the user. The system further determine a bladder status based on the EIT image of the user and predict an incontinence risk based on the bladder status, the posture, and the planning activity of the user and determine a high-risk point indicating a high incontinence risk. Then the system may notify the user or a caregiver of the high incontinence risk at the high-risk point. More specifically, the EIT may detect changes in electric impedance caused by urine or fecal matter, providing a non-invasive way to detect bladder status in real-time. This information may be combined with data from the posture detector and incontinence event detector to provide a more comprehensive picture of the patient's incontinence patterns and triggers. Further, the posture detector may track the patient's body position and movement, which can be useful in identifying patterns or triggers for incontinence. For example, it may detect that a patient is more likely to experience incontinence when lying down or after certain activities, such as exercise. Thirdly, the incontinence event detector may confirm when a high risk incontinence event has occurred and provide additional context about the event, such as the volume and duration of the leakage.

Overall, the advantage of a wearable incontinence predictor system that includes an EIT, posture detector, and incontinence event detector is that it provides a more comprehensive and accurate picture of a patient's incontinence patterns and triggers. This can lead to more personalized and effective treatment plans, improved patient outcomes, and better quality of life for patients dealing with incontinence.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

Further aspects of the embodiments described herein are provided by the subject matter of the following clauses:

A system for incontinence prediction, comprising: an electrical impedance tomography (EIT) device comprising a plurality of electrodes configured to be worn at a lower abdomen of a user around the bladder, wherein a first two or more electrodes are configured to apply alternating currents to a body of the user, and a second two or more electrodes record resulting potentials detected in the body of the user; and a posture detector, wherein a controller communicatively coupled to the EIT device and the posture detector is configured to: collect an EIT image generated by the EIT device, a posture generated by the posture detector, and a planning activity of the user; determine a bladder status based on the EIT image of the user; predict an incontinence risk based on the bladder status, the posture, and the planning activity of the user and determine a high-risk point indicating a high incontinence risk; and notify the user or a caregiver of the high incontinence risk at the high-risk point.

The system according to any previous clause, wherein the bladder status comprises a non-full status and a full status.

The system according to any previous clause, wherein the non-full status comprises an empty status, a half-full status, and a three-fourths full status.

The system according to any previous clause, wherein the full status is based on a maximum bladder tolerance of the user.

The system according to any previous clause, wherein the maximum bladder tolerance of the user corresponds to a largest liquid volume in the user bladder based on collected historical EIT images since an initial usage of the EIT device by the user.

The system according to any previous clause, wherein the system further comprises an incontinence event detector to identify truth of an incontinence event and validate the predicted incontinence risk.

The system according to any previous clause, wherein the incontinence event detector is attached to a diaper or a pad.

The system according to any previous clause, wherein the incontinence detector is a moisture sensor, a thermistor, or a resistance meter.

The system according to any previous clause, wherein the system further comprises an incontinence prediction module that is pre-trained with pre-training posture-status data comprising posture data indicating subject postures, EIT image data indicating subject bladder statuses, and incontinence data indicating subject incontinence events.

The system according to any previous clause, wherein the pre-training is conducted by modeling dependence between the subject postures, the subject bladder statuses, and truth of the subject incontinence events.

The system according to any previous clause, the system further comprises an incontinence event detector to identify truth of an incontinence event, wherein the incontinence prediction module is validated with historical data comprising user postures, EIT images, and truth of user incontinence events generated since an initial usage of the EIT device by the user.

The system according to any previous clause, wherein the validation data is continuously updated with collected data of the user postures, the EIT images, and the truth of user incontinence events.

The system according to any previous clause, wherein the incontinence prediction module weighs the validation data more than the pre-training posture-status data.

The system according to any previous clause, wherein the electrodes are allocated in a band shape for 2D EIT imaging or in a multiple-array structure for 3D EIT imaging.

The system according to any previous clause, wherein the alternating currents are small signal currents.

The system according to any previous clause, wherein the alternating currents have amplitudes below 1 mA and frequency between 1 kHz to 100 kHz.

The system according to any previous clause, wherein the EIT device further comprises a AC/DC converter and an analog-to-digital converter to generate real parts and image parts of the impedance.

The system according to any previous clause, wherein the EIT device measures a bladder impedance corresponding to liquid volume in the bladder and a background impedance corresponding to background tissues.

The system according to any previous clause, wherein the background impedance is constant and the bladder impedance changes according to liquid volume in the bladder.

The system according to any previous clause, wherein the bladder impedance change is linear with the liquid volume in the bladder.

The system according to any previous clause, wherein the background tissues include pelvic internal organs, muscle, and fat of the user.

The system according to any previous clause, wherein the posture comprises a sitting posture, a standing posture, a flat laying posture, an inclined laying posture, or a side laying posture.

The system according to any previous clause, wherein the posture detection sensor is an unobtrusive flexible sensor, an accelerometer, or a gyroscope.

The system according to any previous clause, wherein the posture detection sensor is worn around knees, hips, or thighs of the user.

The system according to any previous clause, wherein the controller is further configured to detect dehydration of the user based on the bladder status and transmits an alarm to a dashboard or a nurse-call system.

The system according to any previous clause, wherein the controller is further configured to transmit the bladder status and the incontinence risk to an external infusion pump, where the external infusion pump adjusts flow rates of fluids and nutrients to the user based on the bladder status or the incontinence risk.

The system according to any previous clause, wherein the controller is further configured to transmit the bladder status and the incontinence risk to an external female catheter, where the external female catheter adjusts vacuum pump output based on the bladder status or the incontinence risk.

The system according to any previous clause, further comprising the controller.

A method for incontinence prediction, comprising: receiving pre-training posture-status data comprising posture data indicating subject postures, EIT image data indicating subject bladder statuses, and incontinence data indicating subject incontinence events; training an incontinence prediction module with the pre-training posture-status data by modeling dependence between the subject postures, the subject bladder statuses, and truth of the subject incontinence events; collecting validation data comprising historical data comprising user postures, EIT images, and truth of user incontinence events generated since an initial usage of the EIT device by a user; validating and updating the incontinence prediction module with the validation data; collecting an EIT image, a posture, and a planning activity of the user; determining a bladder status based on the EIT images of the user; predicting an incontinence risk based on the bladder status, the posture, and the planning activity of the user and determining a high-risk point indicating a high incontinence risk; and notifying the user or a caregiver of the high incontinence risk at the high-risk point.

The method according to any previous clause, wherein the bladder status comprises a full status, an empty status, a half-full status, and a three-fourth full status.

The method according to any previous clause, wherein the full status is based on a maximum bladder tolerance of the user corresponding to a largest liquid volume in the user bladder detected based on collected historical EIT images since an initial usage of the EIT device by the user.

The method according to any previous clause, wherein the validation data are weighed more than the pre-training posture-status data.

The method according to any previous clause, wherein the method further comprises detecting dehydration of the user based on the bladder status and transmitting an alarm to a dashboard or a nurse-call system.

The method according to any previous clause, wherein the method further comprises transmitting the bladder status and the incontinence risk to an external infusion pump, where the infusion pump adjusts flow rates of fluids and nutrients to the user based on the bladder status or the incontinence risk.

The method according to any previous clause, wherein the method further comprises transmitting the bladder status and the incontinence risk to an external female catheter, where the external female catheter adjusts vacuum pump output based on the bladder status or the incontinence risk.

An garment for incontinence prediction comprising: a garment body formed of a flexible fabric, wherein the garment body is configured to be worn on a lower torso of a user; an electrical impedance tomography (EIT) device comprising a plurality of electrodes arranged at discrete locations on the garment body, wherein each electrode is positioned on an inner surface of the garment body and is configured to contact at a lower abdomen of the user around the bladder; a posture detector arranged at an inner surface of the garment body; and a controller communicatively coupled to the EIT device and the posture detector, the controller configured to: collect an EIT image generated by the EIT device, a posture generated by the posture detector, and a planning activity of the user; determine a bladder status based on the EIT image of the user; predict an incontinence risk based on the bladder status, the posture, and the planning activity of the user and determine a high-risk point indicating a high incontinence risk; and notify the user or a caregiver of the high incontinence risk at the high-risk point.

The garment according to any previous clause, wherein the plurality of electrodes of the EIT device comprises a first two or more electrodes are configured to apply alternating currents to a body of the user, and a second two or more electrodes record resulting potentials detected in the body of the user, wherein the electrodes are allocated in a belt shape for 2D EIT imaging or in a multiple-array structure for 3D EIT imaging.

The garment according to any previous clause, wherein the article of clothing comprises a pair of undergarments, where the pair of undergarments is a pair of boxers, a pair of briefs, a pair of boxer briefs, a pair of trunks, a pair of midway briefs, or a pair of thermal underwear.

The garment according to any previous clause, wherein the bladder status comprises an empty status, a half-full status, a three-fourths full status, and a full status.

The garment according to any previous clause, wherein the full status is based on a maximum bladder tolerance of the user, the maximum bladder tolerance of the user corresponds to a largest liquid volume in the user bladder based on collected historical EIT images since an initial usage of the EIT device by the user.

The garment according to any previous clause, wherein the controller further comprises an incontinence prediction module that is pre-trained with pre-training posture-status data comprising posture data indicating subject postures, EIT image data indicating subject bladder statuses, and incontinence data indicating subject incontinence events.

The garment according to any previous clause, wherein the pre-training is conducted by modeling dependence between the subject postures, the subject bladder statuses, and truth of the subject incontinence events.

The garment according to any previous clause, wherein the garment further comprises an incontinence event detector arranged at an inner surface of the garment body to identify truth of an incontinence event and validate the predicted incontinence risk, wherein the incontinence detector is a moisture sensor, a thermistor, or a resistance meter.

The garment according to any previous clause, wherein the incontinence prediction module is validated with historical data comprising user postures, EIT images, and truth of user incontinence events generated since an initial usage of the EIT device by the user.

The garment according to any previous clause, wherein the incontinence prediction module weighs the validation data more than the pre-training posture-status data.

The garment according to any previous clause, wherein the posture comprises a sitting posture, a standing posture, a flat laying posture, an inclined laying posture, or a side laying posture.

The garment according to any previous clause, wherein the posture detection sensor is an unobtrusive flexible sensor, an accelerometer, or a gyroscope.

A method for incontinence prediction, comprising: collecting, by a controller, an EIT image generated by the EIT device, a posture generated by the posture detector, and a planning activity of a user, wherein the EIT device comprises a plurality of electrodes configured to be worn at a lower abdomen of the user around a bladder, wherein a first two or more electrodes are configured to apply alternating currents to a body of the user, and a second two or more electrodes record resulting potentials detected in the body of the user; determining, by the controller, a bladder status based on the EIT image of the user; predicting, by the controller, an incontinence risk based on the bladder status, the posture, and the planning activity of the user and determine a high-risk point indicating a high incontinence risk; and notifying, by the controller, the user or a caregiver of the high incontinence risk at the high-risk point.

The method according to any previous clause, further comprising: detecting, by the controller, dehydration of the user based on the bladder status and transmit an alarm to a dashboard or a nurse-call system.

The method according to any previous clause, further comprising: transmitting, by the controller, the bladder status and the incontinence risk to an external infusion pump, where the external infusion pump adjusts flow rates of fluids and nutrients to the user based on the bladder status or the incontinence risk.

The method according to any previous clause, further comprising: transmitting, by the controller, the bladder status and the incontinence risk to an external female catheter, where the external female catheter adjusts vacuum pump output based on the bladder status or the incontinence risk.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

What is claimed is:

1. A system for incontinence prediction, comprising:
an electrical impedance tomography (EIT) device comprising a plurality of electrodes configured to be worn at a lower abdomen of a user around a bladder, wherein a first two or more electrodes are configured to apply alternating currents to a body of the user, and a second two or more electrodes record resulting potentials detected in the body of the user; and
a posture detector, wherein one or more processors communicatively coupled to the EIT device and the posture detector is configured to:
collect an EIT image generated by the EIT device, a posture generated by the posture detector, and a planning activity of the user;
determine a bladder status based on the EIT image of the user;
predict an incontinence risk based on the bladder status, the posture, and the planning activity of the user and determine a high-risk point indicating a high incontinence risk; and
notify the user or a caregiver of the high incontinence risk at the high-risk point.

2. The system of claim 1, wherein the bladder status comprises a an empty status, a half-full status, a three-fourths full status, and a full status, wherein the full status is based on a maximum bladder tolerance of the user, wherein the maximum bladder tolerance of the user corresponds to a largest liquid volume in the bladder based on collected historical EIT images since an initial usage of the EIT device by the user.

3. The system of claim 1, wherein the system further comprises an incontinence event detector to identify truth of an incontinence event and validate the predicted incontinence risk, wherein the incontinence event detector is attached to a diaper or a pad, and the incontinence event detector is a moisture sensor, a thermistor, or a resistance meter.

4. The system of claim 1, wherein the system further comprises an incontinence prediction module that is pre-trained with pre-training posture-status data comprising posture data indicating subject postures, EIT image data indicating subject bladder statuses, and incontinence data indicating subject incontinence events.

5. The system of claim 4, wherein the pre-training is conducted by modeling dependence between the subject postures, the subject bladder statuses, and truth of the subject incontinence events.

6. The system of claim 4, the system further comprises an incontinence event detector to identify truth of an incontinence event, wherein the incontinence prediction module is validated with historical data comprising user postures, EIT images, and truth of user incontinence events generated since an initial usage of the EIT device by the user.

7. The system of claim 1, wherein the EIT device further comprises a AC/DC converter and an analog-to-digital converter to generate real parts and image parts of electrical impedance.

8. The system of claim 1, wherein the EIT device measures a bladder impedance corresponding to liquid volume in the bladder and a background impedance corresponding to background tissues, wherein the background impedance is constant and the bladder impedance changes according to liquid volume in the bladder.

9. The system of claim 1, wherein the posture comprises a sitting posture, a standing posture, a flat laying posture, an inclined laying posture, or a side laying posture.

10. The system of claim 1, wherein the posture detector is configured to be worn around knees, hips, or thighs of the user.

11. An garment for incontinence prediction, comprising:
a garment body formed of a flexible fabric, wherein the garment body is configured to be worn on a lower torso of a user;
an electrical impedance tomography (EIT) device comprising a plurality of electrodes arranged at discrete locations on the garment body, wherein each electrode is positioned on an inner surface of the garment body and is configured to contact at a lower abdomen of the user around a bladder;
a posture detector arranged at the inner surface of the garment body; and
one or more processors communicatively coupled to the EIT device and the posture detector, the one or more processors configured to:
collect an EIT image generated by the EIT device, a posture generated by the posture detector, and a planning activity of the user;
determine a bladder status based on the EIT image of the user;
predict an incontinence risk based on the bladder status, the posture, and the planning activity of the user and determine a high-risk point indicating a high incontinence risk; and
notify the user or a caregiver of the high incontinence risk at the high-risk point.

12. The garment of claim 11, wherein the plurality of electrodes of the EIT device comprises a first two or more electrodes are configured to apply alternating currents to a body of the user, and a second two or more electrodes record resulting potentials detected in the body of the user, wherein the electrodes are allocated in a belt shape for 2D EIT imaging or in a multiple-array structure for 3D EIT imaging.

13. The garment of claim 11, wherein the garment body comprises a pair of undergarments, where the pair of undergarments is a pair of boxers, a pair of briefs, a pair of boxer briefs, a pair of trunks, a pair of midway briefs, or a pair of thermal underwear.

14. The garment of claim 11, wherein the bladder status comprises an empty status, a half-full status, a three-fourths full status, and a full status, wherein the full status is based on a maximum bladder tolerance of the user, the maximum bladder tolerance of the user corresponds to a largest liquid volume in the bladder based on collected historical EIT images since an initial usage of the EIT device by the user.

15. The garment of claim 11, wherein the one or more processors further comprises an incontinence prediction module that is pre-trained with pre-training posture-status data comprising posture data indicating subject postures, EIT image data indicating subject bladder statuses, and incontinence data indicating subject incontinence events.

16. The garment of claim 15, wherein the pre-training is conducted by modeling dependence between the subject postures, the subject bladder statuses, and truth of the subject incontinence events.

17. The garment of claim 15, wherein the garment further comprises an incontinence event detector arranged at the inner surface of the garment body to identify truth of an incontinence event and validate the predicted incontinence risk, wherein the incontinence event detector is a moisture sensor, a thermistor, or a resistance meter.

18. The garment of claim 17, wherein the incontinence prediction module is validated with historical data comprising user postures, EIT images, and truth of user incontinence events generated since an initial usage of the EIT device by the user.

19. The garment of claim 11, wherein the posture comprises a sitting posture, a standing posture, a flat laying posture, an inclined laying posture, or a side laying posture.

20. A method for incontinence prediction, comprising:
collecting, by one or more processors, an electrical impedance tomography (EIT) image generated by the EIT device, a posture generated by a posture detector, and a planning activity of a user, wherein the EIT device comprises a plurality of electrodes configured to be worn at a lower abdomen of the user around a bladder, wherein a first two or more electrodes are configured to apply alternating currents to a body of the user, and a second two or more electrodes record resulting potentials detected in the body of the user;
determining, by the one or more processors, a bladder status based on the EIT image of the user;
predicting, by the one or more processors, an incontinence risk based on the bladder status, the posture, and the planning activity of the user and determine a high-risk point indicating a high incontinence risk; and
notifying, by the one or more processors, the user or a caregiver of the high incontinence risk at the high-risk point.
